(19) United States
(12) Patent Application Publication
(10) Pub. No.: US 2007/0110705 A1
(43) Pub. Date: May 17, 2007

(54) Composition comprising urginea maritima or drimia maritima, laurus nobilis, adiantum capillus-veneris, and persea americana and its use against hair greasiness, hair loss and for promoting hair growth

(76) Inventor: Emmanouel Marinos, Athens (GR)

Correspondence Address: CHRISTOPHER & WEISBERG, P.A., 200 EAST LAS OLAS BOULEVARD, SUITE 2040, FORT LAUDERDALE, FL, 33301, US

(21) Appl. No.: 10/581,581

(22) PCT Filed: Dec. 3, 2004
(86) PCT No.: PCT/GR04/00057

§ 371 Date: Jun. 2, 2006

(30) Foreign Application Priority Data
Dec. 5, 2003 (GB) 20030100511

(51) Int. Cl. A61K 8/97; A61K 36/73; A61K 36/54

(52) U.S. Cl. 424/74; 424/769; 424/765

(57) ABSTRACT

A main active mixture in liquid form or powder or cream form, prepared by the plants urginea maritime or drimia maritime, laurus nobilis, adiantum capillus-veneris, persea americana and also by distilled distilled pure petroleum, appropriate for dermatological use. The above main active mixture can be mixed with any liquid soap and used as a liquid hair washing or clearance product (shampoo), or used in its simple form, diluted with any essence or oil, preferably distillated, appropriate for dermatological use, as a liquid hair fortifier product (lotion). The main active mixture, the hair washing and, clearance product and the hair fortifier product are used against greasiness, sebum, hair loss and result in hair growth.

### FIELD OF THE INVENTION

The present invention relates to a preparation for the hair in liquid form or powder or cream form, composed by substances produced by plants and optionally distilled pure petroleum, especially for use against the greasiness of the scalp reducing hair loss and resulting in hair growth.

In particular, the preparation according to the present invention consists of substances produced by the following plants by any means (infusion, extraction, distillation): urginea maritime or drimia maritime, laurus nobilis, adiantum capillus-veneris, persea americana, and optionally distilled pure petroleum, appropriate for dermatological use. From said plants, mixed in the suggested proportions and percentages, the main active liquid or powder or cream mixture is prepared, which then can be mixed with any kind of liquid soap to be used as a hair washing and clearance product (shampoo), or alternatively, the main mixture can be used in its simple form, diluted with an essence or distilled pure petroleum, preferably appropriately distilled for dermatological use, as a hair fortifier product (lotion).

### BACKGROUND OF THE INVENTION

Various mixtures derived from plants or chemicals have been used in order to reduce hair loss or for particular hair treatments, for example against pitiriasis or dandruff or dry hair. However, no active mixture has been produced, from which a hair washing and clearance product (shampoo) and hair fortifier product (lotion) is prepared and most importantly, which acts simultaneously against hair greasiness and sebum, to stop hair loss and result in hair growth.

### SUMMARY OF THE INVENTION

The novelty of the present invention is that, as found by the inventor, the liquid form or powder or cream form made by the above-mentioned plants optionally together with distilled pure petroleum and the mixture thereof, achieves the above beneficial action of the main active mixture.

An advantage of the present invention is that the preparation, used as a main active mixture, when mixed with a liquid hair washing product the (shampoo) or a liquid hair fortifier product (lotion), significantly reduces hair loss and results in hair growth.

Said preparation is very useful and practical, destined for use in the hair and the scalp of men and women, revitalizing and preserving their health and beauty in a natural way.

A preferred embodiment of the present invention is a liquid preparation, consisting of a main active mixture used for the preparation of a hair washing fortifier product or a hair fortifier lotion product, is produced under the following procedure: in the beginning the above plants are added and mixed in the following approximate and preferable percentages and in proportion to the general quantity of the active mixture we wish to prepare, as follows: 25% of urginea maritime or drimia maritime, 20% of laurus nobilis, 20% of adiantum capillus-veneris, 25% of persea americana. In said mixture distilled distilled pure petroleum is added, appropriate for dermatological use in the percentage of 10% of the total quantity of the active mixture prepared. The addition of distilled pure petroleum is optional, however, stronger action of the mixture is achieved and the pores of the skin can more easily expel the useless substances aggregated in the skin such as sebum, greasiness and various types of dirt.

In case distilled pure petroleum is not added, the above percentages of each plant must be increased to an additional 2.5%.

In this way the main active mixture is prepared, the action of which is specific, when it is used on the scalp. It acts against greasiness and sebum, strengthens the hair and the scalp, and as a result the hair loss is significantly decreased whilst hair growth is caused helping the hair to grow naturally.

The advisable mixture proportion, in percentages, of the main active mixture and the liquid soap (shampoo) for the preparation of the liquid hair washing product is the following: 2 to 10% of the main active mixture and the remaining percentage should be completed with 100% common liquid soap (shampoo). By increasing or decreasing the above percentages of active mixture, the liquid hair washing product becomes more drastic or less drastic.

For the preparation of a hair fortifier product (lotion) the following proportions must be used: One (1) part of active mixture is mixed with one (1) to five (5) parts of dilution substances, which can be an essence originating from Persea Americana plant or distilled pure petroleum, preferably distilled, appropriate for dermatological use.

In cases where the above proportions differentiate (i.e. if the percentage of a used substance is increased or decreased) in any way, there are no drastic changes in the product's action, provided there is no omission of any of the required substances.

The essential action of the above substances is based on the combined action thereof.

### EXAMPLE 1

In order for 100 ml of main active mixture to be prepared, 25 ml of urginea maritime or drimia maritime, 20 ml of laurus nobilis, 20 ml of adiantum capillus-veneris, 25 ml of persea americana, and optionally 10 ml of distilled pure petroleum preferably distilled, are added and mixed. In case distilled pure petroleum is not added, the above percentages are increased to an additional 2.5% each.

### EXAMPLE 2

In order for 100 ml of liquid hair washing product to be prepared, 10 ml of the main active mixture (which constitutes the 10% of the total quantity of liquid hair washing product) and 90 ml of liquid soap are mixed.

### EXAMPLE 3

In order for 200 ml of liquid hair fortifier product to be prepared, 50 ml of the main active mixture (which constitutes one part of the total quantity of liquid hair fortifier product) and 150 ml of essence originating from the persea americana plant (which constitutes three parts of the total quantity of liquid hair fortifier product) are mixed.

The method of use of the composition according to the invention as a shampoo is to apply it to the scalp and leave it on for 10 to 15 minutes to become activated. Then the scalp should be wet with water and gently massaged. As soon as foam is created the scalp should be washed with water.

The composition used as a lotion is applied in a small quantity to the scalp and left for approximately 10 minutes before it is washed with the shampoo made by the composition according to the invention. The lotion can be separately used for fortification of the hair.

1. A composition suitable for washing, cleaning or treating hair comprising extract, infusion, essence, tincture or pulp of the plants: urginea maritime or drimia maritime, laurus nobilis, adiantum capillus-veneris, persea americana.

2. The composition of claim 1 additionally comprising distilled pure petroleum appropriate for dermatological use.

3. The composition of claims 1 or 2 additionally comprising one or more surfactants selected from the groups comprising soap, anionics, nonionics, cationics amphoterics and zwitterionics.

4. The composition of claims 1, 2 or 3 additionally comprising solvents.

5. The use of extract, infusion, essence, tincture or pulp of the plants: urginea maritime or drimia maritime, laurus nobilis, adiantum capillus-veneris, persea americana, in compositions for washing cleaning or treating hair.

6. A process for reducing hair greasiness and sebum using the composition of claims 1-4.

7. A process for decreasing hair loss using the composition of claims 1-4.

8. A process for regenerating and/or stimulating hair growth using the composition of claims 1-4.

9. A method of preparation of the composition of claims 1-4 wherein the extract, infusion, essence, tincture or pulp of the plants: urginea maritime or drimia maritime, laurus nobilis, adiantum capillus-veneris, persea americana, are mixed in any proportion.

10. The method of claim 9 wherein the extract, infusion, essence, tincture or pulp of the plants: urginea maritime or drimia maritime, laurus nobilis, adiantum capillus-veneris, persea americana are mixed with distilled pure petroleum appropriate for dermatological use and/or one or more surfactants selected from the groups comprising soap, anionics, nonionics, cationics amphoterics and zwitterionics and or solvents.
